(19) United States
(12) Patent Application Publication
(10) Pub. No.: US 2010/0285147 A1
(43) Pub. Date: Nov. 11, 2010

(54) CRUDE DRUG EXTRACT-BLENDED LIQUID MEDICINE

(75) Inventors: Soko Motoune, Hiroshima (JP); Mizuho Ono, Hiroshima (JP); Junichiro Wakamatsu, Hiroshima (JP); Shiho Kano, Hiroshima (JP); Yoichi Ikeda, Hiroshima (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: WAKUNAGA PHARMACEUTICAL CO. LTD, Osaka-shi (JP)

(21) Appl. No.: 12/671,865

(22) PCT Filed: Jul. 31, 2008
(86) PCT No.: PCT/JP2008/002054

§ 371 Date: Feb. 2, 2010

(30) Foreign Application Priority Data
Aug. 3, 2007 (JP) 2007-202590

(51) Int. Cl. A61K 35/12; A61K 36/00; A61K 36/8962; A61K 36/258; A61K 36/16; A61K 35/24; A61K 35/407; A61P 1/00; A61P 43/00

(52) U.S. Cl. 424/537; 424/725; 424/773; 424/754; 424/728; 424/752; 424/520; 424/553

(57) ABSTRACT

Provided an acidic liquid preparation containing a herb and/or animal-origin extract at high concentration to suppress time-dependent occurrence of precipitation and/or opacity.

A liquid preparation containing a herb-origin extract having pH of 3.0 to 5.5 and containing the herb-origin extract at a concentration of 5 to 50 w/v % (dry weight) to the total amount of the liquid preparation, containing a caramel at a concentration of 0.5 to 20 w/v % to the total amount of the liquid preparation, wherein the caramel has isoelectric point of 2.5 to 4.0 and 1.0 w/v % aqueous solution of the caramel has pH of 4.5 to 6.0.

A liquid preparation containing an animal-origin extract having pH of 3.0 to 5.5 and containing the animal-origin extract at concentration of 5 to 50 w/v % (dry weight) to the total amount of the liquid preparation, containing a caramel at concentration of 0.5 to 20 w/v % to the total amount of the liquid preparation, wherein the caramel has isoelectric point of 0.5 to 4.0 and 1.0 w/v % aqueous solution of the caramel has pH of 4.5 to 6.1.

### TECHNICAL FIELD

The present invention relates to a liquid preparation containing a herb and/or animal-origin extract to suppress time-dependent occurrence of precipitation and/or opacity in liquid preparation and to make stable preparation during long-term storage.

### BACKGROUND ART

Natural materials (crude drugs) are wildly used as active ingredient for cold remedies, digestive remedies, and nourishing tonics etc. Among these remedies, a liquid preparation is popularly marketed. Since a liquid preparation containing crude drug extract is easy to ingest and rapidly absorbed to be effective, it is one of preferable forms in pharmaceutical fields. The liquid preparation is commonly taken at a dose of 25 mL to 100 mL, however a reduction of dosage could be desirable for senior persons, children and persons who are weak constitution associated with illness. In order to reduce a dosage with guaranteeing effectiveness of the crude drug, the preparation should be formulated with a large amount of the crude drug extract. However, increase of the crude drug content in the liquid preparation can cause aggregation of constituents and time-dependent increase in precipitation and opacity. These changes could result in decrease of pharmacological activities of the crude drug.

In order to maintain flavors and preserve from microbial growth, lower pH can be essential for the liquid preparation containing the crud drug extract. Contrary, lower pH has been shown to time-dependently increase in precipitation and opacity so that these undesirable changes result in user's discomfort and degradation of product values, and influence effectiveness of the crude drug. These changes could be accelerated with increasing the content of the crude drug extract in the liquid preparation. In conclusion, it is necessary to develop the liquid preparation containing the crude drug extract in high concentration at lower pH without occurrence of precipitation.

Surfactants which enhance dissolubility, for example, have been widely used to suppress time-dependent increase in precipitant and opacity in the liquid preparation containing the crude drug extract. However, use of surfactants is limited because significant amounts of surfactants are required to dissolve a large concentration of the crude drug and lower pH causes hydrolysis of surfactants and decreases abilities thereof.

The present inventors have reported that the occurrence of precipitation in a liquid preparation containing 5 to 50 w/v % of a crude drug extract (dry weight) was suppressed by adding 5 to 40 w/v % of saccharides and adjusting pH at 4.5 to 5.5 (Patent Document 1). Since the precipitation could not be completely suppressed under the lower pH, further pharmaceutical engineering innovations have been required.

Moreover, in a liquid preparation containing an animal-origin extract, the occurrence of precipitation due to animal proteins has been reported to be prevented by adding caramel (Patent Document 2). However, in this report, the preventive effect on occurrence of precipitation was determined in the liquid preparation containing the animal-origin extract at low concentration but not at high concentration.

Patent Document 1: WO 2003/024466

Patent Document 2: JP-A 08-198762

### DISCLOSURE OF THE INVENTION

### Problem to be Solved by the Invention

The present invention is relates to provide an acidic liquid preparation containing a herb and/or animal-origin extract at high concentration, in which the time-dependent occurrence of precipitation or opacity is suppressed.

### Means for Solving Problem

The present inventors conducted an investigation on the prevention of precipitation in an acidic liquid preparation containing a herb and/or animal-origin extract at high concentration and found that the occurrence of precipitation and/or opacity over time can be suppressed, in case where a liquid preparation containing a herb-origin extract is obtained by adding thereto a specific amount of caramel which has isoelectric point of 2.5 to 4.0 and of which 1.0 w/v % aqueous solution has a pH of 4.5 to 6.0 and in case where a liquid preparation containing an animal-origin extract is obtained by blending thereto a specific amount of caramel which has isoelectric points of 0.5 to 4.0 and of which 1.0 w/v % aqueous solution has pH of 4.5 to 6.1.

That is, the invention relates to the following inventions.

1) A liquid preparation containing a herb-origin extract having pH of 3.0 to 5.5 and containing the herb-origin extract at concentration of 5 to 50 w/v % (dry weight) to the total amount of the liquid preparation, containing a caramel at concentration of 0.5 to 20 w/v % to the total amount of the liquid preparation, wherein the caramel has isoelectric point of 2.5 to 4.0 and 1.0 w/v % aqueous solution of the caramel has pH of 4.5 to 6.0.

2) The liquid preparation as set forth above-item 1), wherein the herb-origin extract includes one or more extracts selected from those extracted from Epimedium grandiflorum, Acanthopanax senticosus, Poria sclerotium, Rehmannia Root, Polygonum Root, Cuscuta Seed, Garlic, Ginseng and Ginkgo.

3) The liquid preparation as set forth above-item 1) or 2), containing an animal-origin extract at percentage between 5 to 60% to the herb-origin extract.

4) The liquid preparation as set forth any one of above-items 1) to 3), wherein the herb-origin extract includes at least Garlic extract.

5) The liquid preparations as set forth any one of above-items 1) to 4), further containing a monosaccharide and/or sugar alcohol at concentration between 1.0 and 40 w/v % to the total amount of the liquid preparation.

6) A method of stabilizing a liquid preparation containing a herb-origin extract having pH of 3.0 to 5.5 and containing the herb-origin extract at concentration of 5 to 50 w/v % (dry weight) to the total amount of the liquid preparation, including a caramel at concentration of 0.5 to 20 w/v % to the total amount of the liquid preparation, wherein the caramel has isoelectric point of 2.5 to 4.0 and 1.0 w/v % aqueous solution of the caramel has pH of 4.5 to 6.0.

7) A liquid preparation containing an animal-origin extract having pH of 3.0 to 5.5 and containing the animal-origin extract at concentration of 5 to 50 w/v % (dry weight) to the total amount of the liquid preparation; containing a caramel at concentration of 0.5 to 20 w/v % to the total amount of the liquid preparation, wherein the caramel has isoelectric point of 0.5 to 4.0 and a 1.0 w/v % aqueous solution of the caramel has pH of 4.5 to 6.1.

8) The liquid preparations as set forth above-item 7), wherein the animal-origin extract includes at least one or more extracts selected from those extracted from Oriental Bezoar, Civet, Royal Jelly, Deer Velvet Antler and a liver hydrolysate.

9) The liquid preparations as set forth above-item 7) or 8), further containing a monosaccharide and/or sugar alcohol at concentrations between 1.0 and 40 w/v % to the total amount of the liquid preparation.

10) A method of stabilizing a liquid preparation containing an animal-origin extract containing having pH of 3.0 to 5.5 and containing the animal-origin extract at concentration of 5 to 50 w/v % (dry weight) to the total amount of the liquid preparation, including a caramel at concentration of 0.5 to 20 w/v % to the total amount of the liquid preparation, wherein the caramel has isoelectric point of 0.5 to 4.0 and a 1.0% w/v aqueous solution of the caramel has pH of 4.5 to 6.1.

### EFFECT OF THE INVENTION

The liquid preparation containing the herb and/or animal-origin extract according to the present invention is stable since the occurrence of precipitation or turbidity over time caused by the herbal and/or animal material is prevented, and thus can be taken up without causing an unpleasant feeling to the consumers.

### BEST MODES FOR CARRYING OUT THE INVENTION

A herb-origin extract used in a liquid preparation containing a herb-origin extract according to the present invention refers to the whole or a part of a naturally occurring vegetable material that can be provided for medicinal uses, either directly or after processing. Examples of the herb-origin extract include products described in official compendium of the Japanese Pharmacopoeia and the like or other general-purpose herb-origin extract, such as Mallotus Bark, Gambir, Aloe, Epimedium grandiflorum, Fennel, Mume Fructus, Lindera Root, Bearberry Leaf, Turmeric, Rose Fruit, Acanthopanax senticosus, Corydalis Tuber, Plectranthi Herba, Astragalus Root, Scutellaria Root, Polygonatum Rhizome, Phellodendron Bark, Pruni Jamasakura Cortex, Coptis Rhizome, Polygala Root, Polygonum Root, Zedoary, Pueraria Root, Japanese Valerian, Chamomille, Guarana, Glycyrrhiza, Platycodon Root, Immature Orange, Apricot Kernel, Lycium Fruit, Schizonepeta Spike, Cinnamon Bark, Cassia Seed, Gentian, Geranium Herb, Red Ginseng, Magnolia Bark, Acanthopanax Root Bark, Achyrantes Root, Evodia Fruit, Schisandra Fruit, Bupleurum Root, Asiasarum Root, Thyme, Salvia, Smilax Rhizome, Hawthorn, Gardenia Fruit, Cornus Fruit, Zanthoxylum Fruit, Jujube Seed, Dioscorea Rhizome, Rehmannia Root, Peony Root, Cnidium Monnieri Fruit, Plantago Herb, Houttuynia Herb, Amomum Seed, Ginger, Cardamon, Ligustri Fructus, Lumbricus, Magnolia Flower, Senega, Cnidium Rhizome, Peucedanum Root, Swertia Herb, Atractylodes Lancea Rhizome, Mulberry Bark, Perilla Herb, Rhubarb, Jujube, Clove, Uncaria Hook, Citrus Unshiu Peel, Capsicum, Japanese Angelica Root, Codonopsis pilosula, Cordyceps sinensis, Peach Kernel, Bitter Orange Peel, Ipecac, Cuscuta Seed, Eucommia Bark, Nandina Fruit, Corn Silk, Cistanchis Herb, Garlic, Ophiopogon Tuber, Glehnia Root, Pinellia Tuber, Atractylodes Rhizome, Poria Sclerotium, Sinomenium Stem, Psoraleae Semen, Moutan Bark, Hop, Ephedra Herb, Silvervine, Muira Puama, Saussurea Root, Coix Seed, Longan Aril, Japanese Gentian, and Scopolia Rhizome; as well as those herb-origin extract from which efficacy can be expected in particular, such as Chrysanthemum Flower, Wheat's Shoot, Safflower, Salacia, Blueberry, Rosemary, Japanese Honeysuckle, Medicinal Ginseng (Panax notoginseng, Panax ginseng, Panax japonicus, Panax quinquefolium, Panax vietnamensis, Panax zingiberensis, Panax stipuleanatus, Panax pseudo-ginseng subsp. himalaicus Hara), or Ginkgo. Preferred examples include Epimedium grandiflorum, Acanthopanax senticosus, Poria Sclerotium, Rehmannia Root, Polygonum Root, Cuscuta Seed, Garlic, Medicinal Ginseng and Ginkgo, and even more preferred examples include Epimedium grandiflorum, Acanthopanax senticosus, Cuscuta Seed, Garlic and Medicinal Ginseng. Particularly preferred examples are one or more herb-origin extracts containing at least Garlic.

The content of the herb-origin extract in the liquid preparation containing the herb-origin extract is 5 to 50 w/v %, preferably 7 to 45 w/v %, and more preferably 10 to 40 w/v %, in terms of dry weight, based on the total amount of the liquid preparation.

The liquid preparation containing the herb-origin extract above can contain an animal-origin extract shown below. In this case, the animal-origin extract is preferably incorporated at a percentage of 5 to 60% by mass, more preferably 10 to 50% by mass, and more preferably 15 to 40% by mass, based on the herb-origin extract.

Examples of the animal-origin extract in the liquid preparation containing the animal-origin extract according to the present invention include Phocae Thstis et Penis, Seahorse, Oriental Bezoar, Civet, Deer Velvet Antler, Agkistrodon japonica, and the like, as well as Royal Jelly, and animal organs such as liver, heart or placenta. The animal-origin extract includes extracts of those materials, or hydrolysates produced using an acid, a base or an enzyme (liver hydrolysate, brain hydrolysate, placenta extract, heart extract, and the like). Preferred examples of the animal-origin extract include extracts of Oriental Bezoar, Civet and Deer Velvet Antler, Royal Jelly and a liver hydrolysate, and particularly preferred examples include extracts of Oriental Bezoar, Civet and Deer Velvet Antler, and a liver hydrolysate.

The content of the animal-origin extract in the liquid preparation containing the animal-origin extract is 5 to 50 w/v %, preferably 7 to 45 w/v %, and more preferably 10 to 40 w/v %, in terms of dry weight, based on the total amount of the liquid preparation.

Preparation of the herb and/or animal-origin extract may be carried out according to conventional methods. For example, the whole or a part of each herbal and/or animal material is directly, or after drying, fine cutting and grinding, extracted with an appropriate solvent such as water; an alcohol such as methanol, ethanol, 1-propanol, 2-propanol or 1-butanol; and a liquid mixture thereof, and the extract is concentrated and then removed of unnecessary components or the like by known techniques such as appropriate filtration, activated carbon treatment, and silica gel column chromatography. Thereby the herb and/or animal-origin extract can be produced.

The herb and/or animal-origin extract of the present invention includes a viscous extract, a dry extract, a fluid extract, a tincture and the like.

Here, in the case of a tincture or a fluid extract, the amount of the herb and/or animal-origin extract in terms of dry weight refers to the amount of solid content calculated in terms of the evaporation residue, and this can be calculated from the weight of a residue (evaporation residue) obtainable by measuring 10 mL of a tincture or a fluid extract in a beaker with known weight, drying the content by evaporation in a boiling water bath, drying the content at 105° C. for 6 hours, and then leaving the content to cool in a desiccator (containing silica gel). For example, the amount of solid content in 1 mL of a tincture having 300 mg of an evaporation residue is 30 mg. Furthermore, in the case of a viscous extract, the amount in terms of dry weight refers to the amount of solid content calculated in terms of a loss on drying obtained by a test method prescribed in the Japanese Pharmacopoeia. For example, the amount of solid content in 1 g of a viscous extract having a loss on drying of 60% is 400 mg.

The term “caramel” refers to a product obtained by heat treating an edible hydrocarbon such as sugar or glucose, and more particularly means a brown liquid or powder corresponding to any of the following as defined in Japan's Specifications and Standards for Food Additives, 7^{th} Edition (1991) as follows: (I) a product obtained by heat treating an edible hydrocarbon such as a starch hydrolysate, molasses or a saccharide, or obtained by heat treating those materials in the presence of an acid or an alkali, without using a sulfurous acid compound and an ammonium compound; (II) a product obtained by adding a sulfurous acid compound to an edible hydrocarbon such as a starch hydrolysate, molasses or a saccharide, or optionally further adding an acid or an alkali to this, and heat treating the mixture, without using an ammonium compound; (III) a product obtained by adding an ammonium compound to an edible hydrocarbon such as a starch hydrolysate, molasses or a saccharide, or optionally further adding an acid or an alkali to this, and heat treating the mixture, without using a sulfurous acid compound; (IV) a product obtained by adding a sulfurous acid compound and an ammonium compound to an edible hydrocarbon such as a starch hydrolysate, molasses or a saccharide, or optionally further adding an acid or an alkali to this, and heat treating the mixture. However, the caramel used in the present invention is preferably a product produced by the method of (I). Furthermore, the edible hydrocarbon that serves as a raw material is not particularly limited, but saccharides such as sugar, glucose and fructose are preferred.

In regard to the caramel, when the liquid preparation containing the herb-origin extract is produced, a caramel which has isoelectric point in the range of 2.5 to 4.0 and of which 1.0 w/v % aqueous solution has pH of 4.5 to 6.0 is used. A caramel having isoelectric point of 2.8 to 3.1 is more preferred, and a caramel of which 1.0 w/v % aqueous solution has pH of 4.9 to 5.7 is more preferred.

As for such a caramel, commercially available products can be used, and for example, Caramel SF (manufactured by Ikeda Tohka Industries Co., Ltd.), Caramel S-W (manufactured by Semba Tohka Industries Co., Ltd.), Caramel C-85 (manufactured by Amano Jitsugyo Co., Ltd.), Caramel FS (manufactured by Showa Chemical Industry Co., Ltd.), Caramel FS-350S (manufactured by Showa Chemical Industry Co., Ltd.), and the like can be used.

When the liquid preparation containing the animal-origin extract is produced, a caramel which has isoelectric point in the range of 0.5 to 4.0 and of which 1.0 w/v % aqueous solution has pH of 4.5 to 6.1 is used. The isoelectric point may be, for example, in the range of 0.5 to 1.0, 0.5 to 2.4, 0.6 to 4.0, or the like. A caramel having isoelectric point of 0.6 to 3.6 is more preferred, and a caramel of which 1.0 w/v % aqueous solution has pH of 4.9 to 6.1 is more preferred.

As for such a caramel, commercially available products can be used, and for example, Caramel SF (manufactured by Ikeda Tohka Industries Co., Ltd.), Caramel S-W (manufactured by Semba Tohka Industries Co., Ltd.), Caramel KD-W (manufactured by Semba Tohka Industries Co., Ltd.), Caramel BCF-3 (manufactured by Ikeda Tohka Industries Co., Ltd.), Caramel C-85 (manufactured by Amano Jitsugyo Co., Ltd.), Caramel SS (manufactured by Amano Jitsugyo Co., Ltd.), Caramel KS-W (manufactured by Semba Tohka Industries Co., Ltd.), Caramel FS (manufactured by Showa Chemical Industry Co., Ltd.), Caramel FS-350S (manufactured by Showa Chemical Industry Co., Ltd.), Caramel S (manufactured by Ikeda Tohka Industries Co., Ltd.), and the like can be used.

The amount of caramel incorporated into the liquid preparation can be in the range of 0.5 to 20 w/v % to the total amount of the liquid preparation, and the amount is preferably 0.7 to 18 w/v %, and more preferably 0.8 to 15 w/v %.

The liquid preparation containing the herb and/or animal-origin extract according to the present invention can be blended with various efficacious ingredients such as, for example, vitamins such as vitamin B₁'s (thiamine hydrochloride, thiamine nitrate, and the like), vitamin B₂'s, vitamin B₆'s, vitamin E's, nicotinic acids, pantothenic acids, and vitamin B₁₂'s; an antipyretic analgesic, an antihistamine agent, an expectorant, and an antitussive agent insofar as not diminishing the effect of the caramel.

Furthermore, the liquid preparation containing the herb and/or animal-origin extract according to the present invention can be added, if necessary, with a monosaccharide and/or a sugar alcohol for the purpose of assisting dissolution, and the blending amount that can be used is in the range of 1.0 to 40 w/v % based on the total amount of liquid preparation. The amount is preferably 3 to 35 w/v %, and more preferably 5 to 30 w/v %.

Here, examples of the monosaccharide include fructose, fucose, mannose, glucose, galactose, ribose and the like, but particularly fructose, glucose and galactose are preferred. The sugar alcohol that is used in the present invention may be a commercially available product, and examples thereof include erythritol, xylitol, maltitol, mannitol, D-sorbitol, lactitol, glycerol, inositol and mixed liquids thereof. However, sorbitol or xylitol may be preferably used.

These additives can be used, for example, when it is wished to reduce the amount of addition of caramel to the liquid preparation containing the herb and/or animal-origin extract according to the present invention, and when the precipitation preventing effect is diminished by the reduction of the caramel.

In regard to a method for producing the liquid preparation containing the herb and/or animal-origin extract according to the present invention, for example, one or more kinds of herb and/or animal-origin extracts are dissolved in water in an amount in the range of 5 to 50 w/v %, in terms of dry weight, based on the total amount of the finally obtainable liquid preparation, and if necessary, any insoluble matters are removed therefrom by centrifugation, column chromatography or filtration. Caramel is added to the obtained solution in an amount of 5 to 40 w/v % based on the total amount of the finally obtainable liquid preparation, and the mixture is stirred at room temperature. Subsequently, an appropriate pH regulating agent is added, and the pH is adjusted to 3.0 to 5.5. Water is added to make up the total volume, and then the resultant is filtered, as necessary. Thus, the liquid preparation containing the herb and/or animal-origin extract can be obtained.

The pH regulating agent that is used in the present invention may be any generally known agent, but examples include acids such as citric acid, acetic acid, phthalic acid, succinic acid, maleic acid, aspartic acid, adipic acid, glutamic acid, fumaric acid, phosphoric acid, hydrochloric acid, nitric acid, sulfuric acid, tartaric acid, and malic acid; and bases such as potassium hydroxide, sodium hydroxide, calcium hydroxide, potassium hydrogen carbonate, sodium hydrogen carbonate, potassium carbonate, calcium hydrogen carbonate, sodium carbonate, and calcium carbonate.

### EXAMPLES

Hereinafter, the liquid preparation of the present invention will be described in detail by way of Examples.

In all of the Examples and Comparative Examples shown below, the total amount was made up to 100 mL, and the solutions of the Examples and Comparative Examples were filtered, filled in a glass test tube and then sealed with a cap, to be used as samples.

The isoelectric point of caramel was determined by the following method.

<Method for Measuring Isoelectric Point>

Approximately 0.5 g of caramel is dissolved in 50 mL of a 0.5 w/v % tannic acid solution, and the solution is dispensed into nine glass test tubes in an amount of 5 mL each. If the liquid is clear, 0.4% hydrochloric acid or 19.9% hydrochloric acid is added in a cumulatively increasing manner at an increment of 25 μL, and the mixture is thoroughly mixed, so that the turning point between turbidness and clearness is determined by visual inspection. After the solution is stored at room temperature for 24 hours, the solution is observed again by visual inspection. The pH of the turning point between turbidness and clearness is measured, and this pH is designated as the isoelectric point. When the solution obtained after dissolving caramel is already turbid, 0.4% sodium hydroxide or 16.9% sodium hydroxide is added in a cumulatively increasing manner, similarly to the case of hydrochloric acid, and the following operation is carried out. For example, when the solution pH near the turning point between turbidness and clearness is 2.9, 3.0, 3.1 and 3.2, and the solution is turbid at pH of 2.9 and 3.0, while the solution is clear at pH of 3.1 and 3.2, the isoelectric point is 3.0.

### Example A1

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 5.0 g of Caramel SF (manufactured by Ikeda Tohka Industries Co., Ltd.; pH 5.7, isoelectric point 3.1) was added thereto and dissolved. Subsequently, 10% hydrochloric acid was used to adjust the pH to 4.0, and purified water was added to make up a total volume of 100 mL (Sample A1).

### Example A2

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 5.0 g of Caramel S-W (manufactured by Semba Tohka Industries Co., Ltd.; pH 5.1, isoelectric point 3.1) was added thereto and dissolved. Subsequently, 10% hydrochloric acid was used to adjust the pH to 4.0, and purified water was added to make up a total volume of 100 mL (Sample A2).

### Example A3

12.0 g in terms of dry weight of a Garlic viscous extract was added to a suitable amount of purified water at room temperature, and the mixture was sufficiently stirred. 5.0 g of Caramel S-W (manufactured by Semba Tohka Industries Co., Ltd.; pH 5.1, isoelectric point 3.1) was added thereto and dissolved. Subsequently, 10% hydrochloric acid was used to adjust the pH to 4.0, and purified water was added to make up a total volume of 100 mL (Sample A3).

### Example A4

3.0 g in terms of dry weight of an Acanthopanax senticosus viscous extract, 5.0 g in terms of dry weight of a Ginseng viscous extract, and 3.0 g in terms of dry weight of a Cuscuta Seed viscous extract were added to a suitable amount of purified water at room temperature, and the mixture was sufficiently stirred. 5.0 g of Caramel S-W (manufactured by Semba Tohka Industries Co., Ltd.; pH 5.1, isoelectric point 3.1) was added thereto and dissolved. Subsequently, 10% hydrochloric acid was used to adjust the pH to 4.0, and purified water was added to make up a total volume of 100 mL (Sample A4).

### Example A5

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 5.0 g of Caramel C-85 (manufactured by Amano Jitsugyo Co., Ltd.; pH 4.9, isoelectric point 2.9) was added thereto and dissolved. Subsequently, 10% hydrochloric acid was used to adjust the pH to 4.0, and purified water was added to make up a total volume of 100 mL (Sample A5).

### Example A6

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 5.0 g of Caramel FS (manufactured by Showa Chemical Industry Co., Ltd.; pH 5.5, isoelectric point 2.9) was added thereto and dissolved. Subsequently, 10% hydrochloric acid was used to adjust the pH to 4.0, and purified water was added to make up a total volume of 100 mL (Sample A6).

### Example A7

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 5.0 g of Caramel FS-350S (manufactured by Showa Chemical Industry Co., Ltd.; pH 5.7, isoelectric point 2.8) was added thereto and dissolved. Subsequently, 10% hydrochloric acid was used to adjust the pH to 4.0, and purified water was added to make up a total volume of 100 mL (Sample A7).

### Example A8

11.0 g in terms of dry weight of a Ginkgo viscous extract was added to a suitable amount of purified water at room temperature, and the mixture was sufficiently stirred. 5.0 g of Caramel S-W (manufactured by Semba Tohka Industries Co., Ltd.; pH 5.1, isoelectric point 3.1) was added thereto and dissolved. Subsequently, 10% hydrochloric acid was used to adjust the pH to 4.0, and purified water was added to make up a total volume of 100 mL.

### Example A9

20.0 g in terms of dry weight of a Garlic viscous extract was added to a suitable amount of purified water at room temperature, and the mixture was sufficiently stirred. 5.0 g of Caramel S-W (manufactured by Semba Tohka Industries Co., Ltd.; pH 5.1, isoelectric point 3.1) was added thereto and dissolved. Subsequently, 10% hydrochloric acid was used to adjust the pH to 4.0, and purified water was added to make up a total volume of 100 mL.

### Example A10

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 3.0 g of Caramel SF (manufactured by Ikeda Tohka Industries Co., Ltd.; pH 5.7, isoelectric point 3.1) and 20 g of sorbitol were added thereto and dissolved. Subsequently, 10% hydrochloric acid was used to adjust the pH to 4.0, and purified water was added to make up a total volume of 100 mL (Sample A8).

### Example A11

12.0 g in terms of dry weight of a Garlic viscous extract, 4.0 g in terms of dry weight of a Ginseng viscous extract, and 4.0 g in terms of dry weight of a Cuscuta Seed viscous extract were added to a suitable amount of purified water at room temperature, and the mixture was sufficiently stirred. 5.0 g of Caramel SF (manufactured by Ikeda Tohka Industries Co., Ltd.; pH 5.7, isoelectric point 3.1) and 5.0 g of fructose were added thereto and dissolved. Subsequently, 10% hydrochloric acid was used to adjust the pH to 4.0, and purified water was added to make up a total volume of 100 mL.

### Example A12

4.0 g in terms of dry weight of a Garlic viscous extract, 0.5 g in terms of dry weight of a Ginseng viscous extract, 0.5 g in terms of dry weight of a Cuscuta Seed viscous extract, and 1.0 g in terms of dry weight of a liver hydrolysate were added to a suitable amount of purified water at room temperature, and the mixture was sufficiently stirred. 5.0 g of Caramel SF (manufactured by Ikeda Tohka Industries Co., Ltd.; pH 5.7, isoelectric point 3.1) was added thereto and dissolved. Subsequently, 10% hydrochloric acid was used to adjust the pH to 4.0, and purified water was added to make up a total volume of 100 mL.

### Example A13

13.0 g in terms of dry weight of a Garlic viscous extract, and 2.0 g in terms of dry weight of a liver hydrolysate were added to a suitable amount of purified water at room temperature, and the mixture was sufficiently stirred. 1.2 g of Caramel SF (manufactured by Ikeda Tohka Industries Co., Ltd.; pH 5.7, isoelectric point 3.1) and 6.5 g of fructose were added thereto and dissolved. Subsequently, 10% hydrochloric acid was used to adjust the pH to 4.0, and purified water was added to make up a total volume of 100 mL.

### Comparative Example A1

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred and dissolved. Subsequently, 10% hydrochloric acid was used to adjust the pH to 4.0, and purified water was added to make up a total volume of 100 mL (Sample A9).

### Comparative Example A2

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 5.0 g of Caramel Z-100 (manufactured by Semba Tohka Industries Co., Ltd.; pH 3.5, isoelectric point 0.4) was added thereto and dissolved. Subsequently, 10% hydrochloric acid was used to adjust the pH to 4.0, and purified water was added to make up a total volume of 100 mL (Sample A10).

### Comparative Example A3

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 5.0 g of Caramel S (manufactured by Semba Tohka Industries Co., Ltd.; pH 4.3, isoelectric point 1.2) was added thereto and dissolved. Subsequently, 10% hydrochloric acid was used to adjust the pH to 4.0, and purified water was added to make up a total volume of 100 mL (Sample A11).

### Comparative Example A4

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 5.0 g of Caramel KS-W (manufactured by Semba Tohka Industries Co., Ltd.; pH 6.1, isoelectric point 3.6) was added thereto and dissolved. Subsequently, 10% hydrochloric acid was used to adjust the pH to 4.0, and purified water was added to make up a total volume of 100 mL (Sample A12).

### Comparative Example A5

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water, and the mixture was sufficiently stirred. 5.0 g of Caramel FB (manufactured by Showa Chemical Industry Co., Ltd.; pH 4.0, isoelectric point 3.4) was added thereto and dissolved. Subsequently, 10% hydrochloric acid was used to adjust the pH to 4.0, and purified water was added to make up a total volume of 100 mL (Sample A13).

### Comparative Example A6

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water, and the mixture was sufficiently stirred. 5.0 g of Caramel MG-18W (manufactured by Semba Tohka Industries Co., Ltd.; pH 5.3, isoelectric point 6.5) was added thereto and dissolved. Subsequently, 10% hydrochloric acid was used to adjust the pH to 4.0, and purified water was added to make up a total volume of 100 mL (Sample A14).

### Comparative Example A7

8.0 g in terms of dry weight of a Garlic viscous extract, 1.0 g in terms of dry weight of a Ginseng viscous extract, and 1.0 g in terms of dry weight of a Cuscuta Seed viscous extract were added to a suitable amount of purified water at room temperature, and the mixture was sufficiently stirred. 40 g of sorbitol was added thereto and dissolved. Subsequently, 10% hydrochloric acid was used to adjust the pH to 4.0, and purified water was added to make up a total volume of 100 mL (Sample 15A).

### Test Example A1

Samples A1 to A7 and A9 to A14 were used to carry out a storage test at 25° C. for 6 months. The effect of the addition of caramel on the precipitation or opacity over time of the herbal and/or animal materials in these cases was observed. The results are presented in Table 1. As a result, in the liquid preparations containing the herb-origin extracts samples A1 to A7 of the present invention to which Caramel SF, Caramel S-W, Caramel C-85, Caramel FS and Caramel FS-350W had been added, precipitation or opacity was scarcely observed during the storage for 6 months. However, in the sample A9 to which caramel was not added, and the samples A10 to A14 to which Caramel Z-100, Caramel S, Caramel KS-W, Caramel FB and Caramel MG-18W had been added, precipitation or opacity over time was observed.

TABLE 1

| | Blended caramel | | | | | | |
|---|---|---|---|---|---|---|---|
| | | | Iso- | Storage period | | | |
| | | | electric | Begin- | 2nd | 4th | 6th |
| | Grade | pH | point | ning | month | month | month |
| Sample | SF | 5.7 | 3.1 | − | − | − | − |
| A1 | | | | | | | |
| Sample | S-W | 5.1 | 3.1 | − | − | − | − |
| A2 | | | | | | | |
| Sample | S-W | 5.1 | 3.1 | − | − | − | − |
| A3 | | | | | | | |
| Sample | S-W | 5.1 | 3.1 | − | − | − | − |
| A4 | | | | | | | |
| Sample | C-85 | 4.9 | 2.9 | − | − | − | − |
| A5 | | | | | | | |
| Sample | FS | 5.5 | 2.9 | − | − | − | − |
| A6 | | | | | | | |
| Sample | FS- | 5.7 | 2.8 | − | − | − | − |
| A7 | 350S | | | | | | |
| Sample | No addition | | | − | ++ | ++ | ++ |
| A9 | | | | | | | |
| Sample | Z-100 | 3.5 | 0.4 | − | − | + | ++ |
| A10 | | | | | | | |
| Sample | S | 4.3 | 1.2 | − | ++ | ++ | ++ |
| A11 | | | | | | | |
| Sample | KS-W | 6.1 | 3.6 | − | − | + | ++ |
| A12 | | | | | | | |
| Sample | FB | 4.0 | 3.4 | − | − | + | + |
| A13 | | | | | | | |
| Sample | MG- | 5.3 | 6.5 | − | − | + | ++ |
| A14 | 18W | | | | | | |

−: Clear liquid, or negligible precipitation or opacity occurs
+: Precipitation or opacity occurs
++: Conspicuous precipitation or opacity occurs

### Test Example A2

Samples A8 and A15 were used to carry out a storage test at 40° C. for 4 weeks. The effect of the addition of caramel and sorbitol on the precipitation or opacity over time of the herbal materials in these cases was observed. The results are presented in Table 2. As a result, in the liquid preparation containing the herb-origin extract sample A8 of the present invention to which Caramel SF and sorbitol had been added, precipitation or opacity was scarcely observed during the storage for 4 weeks. However, in the sample A15 to which only sorbitol had been added (caramel was not added), precipitation or opacity over time was observed.

TABLE 2

| | Blended caramel | | | | | | | |
|---|---|---|---|---|---|---|---|---|
| | | | Iso- | Addi- | Storage period | | | |
| | | | electric | tion of | Begin- | 1st | 2nd | 4th |
| | Grade | pH | point | sorbitol | ning | week | week | week |
| Sample | SF | 5.7 | 3.1 | 20 g | − | − | − | − |
| A8 | | | | | | | | |

| | | | | | | |
|---|---|---|---|---|---|---|
| Sample | No addition | 40 g | − | − | + | + |
| A15 | | | | | | |

−: Clear liquid, or negligible precipitation or opacity occurs
+: Precipitation or opacity occurs
++: Conspicuous precipitation or opacity occurs

### Example B1

5.0 g in terms of dry weight of a liver hydrolysate was added to a suitable amount of purified water at room temperature, and the mixture was sufficiently stirred. 5.0 g of Caramel SF (manufactured by Ikeda Tohka Industries Co., Ltd.; pH 5.7, isoelectric point 3.1) was added thereto and dissolved. Subsequently, 10% hydrochloric acid was used to adjust the pH to 4.0, and purified water was added to make up a total volume of 100 mL (Sample B1).

### Example B2

5.0 g in terms of dry weight of a liver hydrolysate was added to a suitable amount of purified water at room temperature, and the mixture was sufficiently stirred. 5.0 g of Caramel S-W (manufactured by Semba Tohka Industries Co., Ltd.; pH 5.1, isoelectric point 3.1) was added thereto and dissolved. Subsequently, 10% hydrochloric acid was used to adjust the pH to 4.0, and purified water was added to make up a total volume of 100 mL (Sample B2).

### Example B3

5.0 g in terms of dry weight of a liver hydrolysate was added to a suitable amount of purified water at room temperature, and the mixture was sufficiently stirred. 5.0 g of Caramel KD-W (manufactured by Semba Tohka Industries Co., Ltd.; pH 5.1, isoelectric point 0.9) was added thereto and dissolved. Subsequently, 10% hydrochloric acid was used to adjust the pH to 4.0, and purified water was added to make up a total volume of 100 mL (Sample B3).

### Example B4

5.0 g in terms of dry weight of a liver hydrolysate was added to a suitable amount of purified water at room temperature, and the mixture was sufficiently stirred. 5.0 g of Caramel BCF-3 (manufactured by Ikeda Tohka Industries Co., Ltd.; pH 5.1, isoelectric point 0.6) was added thereto and dissolved. Subsequently, 10% hydrochloric acid was used to adjust the pH to 4.0, and purified water was added to make up a total volume of 100 mL (Sample B4).

### Example B5

5.0 g in terms of dry weight of a liver hydrolysate was added to a suitable amount of purified water at room temperature, and the mixture was sufficiently stirred. 5.0 g of Caramel C-85 (manufactured by Amano Jitsugyo Co., Ltd.; pH 4.9, isoelectric point 2.9) was added thereto and dissolved. Subsequently, 10% hydrochloric acid was used to adjust the pH to 4.0, and purified water was added to make up a total volume of 100 mL (Sample B5).

### Example B6

5.0 g in terms of dry weight of a liver hydrolysate was added to a suitable amount of purified water at room temperature, and the mixture was sufficiently stirred. 5.0 g of Caramel SS (manufactured by Amano Jitsugyo Co., Ltd.; pH 5.9, isoelectric point 3.5) was added thereto and dissolved. Subsequently, 10% hydrochloric acid was used to adjust the pH to 4.0, and purified water was added to make up a total volume of 100 mL (Sample B6).

### Example B7

5.0 g in terms of dry weight of a liver hydrolysate was added to a suitable amount of purified water at room temperature, and the mixture was sufficiently stirred. 5.0 g of Caramel KS-W (manufactured by Semba Tohka Industries Co., Ltd.; pH 6.1, isoelectric point 3.6) was added thereto and dissolved. Subsequently, 10% hydrochloric acid was used to adjust the pH to 4.0, and purified water was added to make up a total volume of 100 mL (Sample B7).

### Example B8

5.0 g in terms of dry weight of a liver hydrolysate was added to a suitable amount of purified water at room temperature, and the mixture was sufficiently stirred. 3.0 g of Caramel SF (manufactured by Ikeda Tohka Industries Co., Ltd.; pH 5.7, isoelectric point 3.1) and 20 g of sorbitol were added thereto and dissolved. Subsequently, 10% hydrochloric acid was used to adjust the pH to 4.0, and purified water was added to make up a total volume of 100 mL (Sample B8).

### Example B9

5.0 g in terms of dry weight of a liver hydrolysate, 3.2 mg in terms of dry weight of a Oriental Bezoar tincture, 4.1 mg in terms of dry weight of a Civet tincture, and 16.7 mg in terms of dry weight of a Deer Velvet Antler fluid extract were added to a suitable amount of purified water at room temperature, and the mixture was sufficiently stirred. 5.0 g of Caramel KS-W (manufactured by Semba Tohka Industries Co., Ltd.; pH 6.1, isoelectric point 3.6) was added thereto and dissolved. Subsequently, 10% hydrochloric acid was used to adjust the pH to 4.0, and purified water was added to make up a total volume of 100 mL.

### Comparative Example B1

5.0 g in terms of dry weight of a liver hydrolysate was added to a suitable amount of purified water at room temperature, and the mixture was sufficiently stirred and dissolved. Subsequently, 10% hydrochloric acid was used to adjust the pH to 4.0, and purified water was added to make up a total volume of 100 mL (Sample B9).

### Comparative Example B2

5.0 g in terms of dry weight of a liver hydrolysate was added to a suitable amount of purified water at room temperature, and the mixture was sufficiently stirred. 5.0 g of Caramel Z-100 (manufactured by Semba Tohka Industries Co., Ltd.; pH 3.5, isoelectric point 0.4) was added thereto and dissolved. Subsequently, 10% hydrochloric acid was used to adjust the pH to 4.0, and purified water was added to make up a total volume of 100 mL (Sample B10).

### Comparative Example B3

5.0 g in terms of dry weight of a liver hydrolysate was added to a suitable amount of purified water at room temperature, and the mixture was sufficiently stirred. 5.0 g of Caramel S (manufactured by Semba Tohka Industries Co., Ltd.; pH 4.3, isoelectric point 1.2) was added thereto and dissolved. Subsequently, 10% hydrochloric acid was used to adjust the pH to 4.0, and purified water was added to make up a total volume of 100 mL (Sample B11).

### Comparative Example B4

5.0 g in terms of dry weight of a liver hydrolysate was added to a suitable amount of purified water at room temperature, and the mixture was sufficiently stirred. 5.0 g of Caramel KS-S (manufactured by Semba Tohka Industries Co., Ltd.; pH 6.2, isoelectric point 3.2) was added thereto and dissolved. Subsequently, 10% hydrochloric acid was used to adjust the pH to 4.0, and purified water was added to make up a total volume of 100 mL (Sample B12).

### Comparative Example B5

5.0 g in terms of dry weight of a liver hydrolysate was added to a suitable amount of purified water at room temperature, and the mixture was sufficiently stirred. 5.0 g of Caramel FB (manufactured by Showa Chemical Industry Co., Ltd.; pH 4.0, isoelectric point 3.4) was added thereto and dissolved. Subsequently, 10% hydrochloric acid was used to adjust the pH to 4.0, and purified water was added to make up a total volume of 100 mL (Sample B13).

### Comparative Example B6

5.0 g in terms of dry weight of a liver hydrolysate was added to a suitable amount of purified water at room temperature, and the mixture was sufficiently stirred. 5.0 g of Caramel MG-18W (manufactured by Semba Tohka Industries Co., Ltd.; pH 5.3, isoelectric point 6.5) was added thereto and dissolved. Subsequently, 10% hydrochloric acid was used to adjust the pH to 4.0, and purified water was added to make up a total volume of 100 mL (Sample B14).

### Comparative Example B7

5.0 g in terms of dry weight of a liver hydrolysate was added to a suitable amount of purified water at room temperature, and the mixture was sufficiently stirred. 40 g of sorbitol was added thereto and dissolved. Subsequently, 10% hydrochloric acid was used to adjust the pH to 4.0, and purified water was added to make up a total volume of 100 mL (Sample B15).

### Test Example B1

Samples B1 to B7 and B9 to B14 were used to carry out a storage test at 25° C. for 6 months. The effect of the addition of caramel on the precipitation or opacity over time of the animal materials in these cases was observed. The results are presented in Table 3. As a result, in the liquid preparations containing the animal-origin extracts samples 1 to 7 of the present invention to which Caramel SF, Caramel S-W, Caramel KD-W, Caramel BCF-3, Caramel C-85, Caramel SS and Caramel KS-W had been added, precipitation or opacity was scarcely observed during the storage for 6 months. However, in the sample B9 to which caramel was not added, and the samples B10 to B14 to which Caramel Z-100, Caramel S, Caramel KS-S, Caramel FB and Caramel MG-18W had been added, precipitation or opacity over time was observed.

TABLE 3

| | Blended caramel | | | | | | |
|---|---|---|---|---|---|---|---|
| | | | Iso- | Storage period | | | |
| | | | electric | Begin- | 2nd | 4th | 6th |
| | Grade | pH | point | ning | month | month | month |
| Sample | SF | 5.7 | 3.1 | − | − | − | − |
| B1 | | | | | | | |
| Sample | S-W | 5.1 | 3.1 | − | − | − | − |
| B2 | | | | | | | |
| Sample | KD-W | 5.1 | 0.9 | − | − | − | − |
| B3 | | | | | | | |
| Sample | BCF-3 | 5.1 | 0.6 | − | − | − | − |
| B4 | | | | | | | |
| Sample | C-85 | 4.9 | 2.9 | − | − | − | − |
| B5 | | | | | | | |
| Sample | SS | 5.9 | 3.5 | − | − | − | − |
| B6 | | | | | | | |
| Sample | KS-W | 6.1 | 3.6 | − | − | − | − |
| B7 | | | | | | | |
| Sample | No addition | | | − | ++ | ++ | ++ |
| B9 | | | | | | | |
| Sample | Z-100 | 3.5 | 0.4 | − | + | + | + |
| B10 | | | | | | | |
| Sample | S | 4.3 | 1.2 | − | + | ++ | ++ |
| B11 | | | | | | | |
| Sample | KS-S | 6.2 | 3.2 | − | + | + | + |
| B12 | | | | | | | |
| Sample | FB | 4.0 | 3.4 | − | ++ | ++ | ++ |
| B13 | | | | | | | |
| Sample | MG- | 5.3 | 6.5 | − | − | − | ++ |
| B14 | 18W | | | | | | |

−: Clear liquid, or negligible precipitation or opacity occurs
+: Precipitation or opacity occurs
++: Conspicuous precipitation or opacity occurs

### Test Example B2

Samples B8 and B15 were used to carry out a storage test at 40° C. for 4 weeks. The effect of the addition of caramel and sorbitol on the precipitation or opacity over time of the animal materials in these cases was observed. The results are presented in Table 4. As a result, in the liquid preparation containing the animal-origin extract sample B8 of the present invention to which Caramel SF and sorbitol had been added, precipitation or opacity was scarcely observed during the storage for 4 weeks. However, in the sample B15 to which only sorbitol had been added (caramel was not added), precipitation or opacity over time was observed.

TABLE 4

| | Blended caramel | | | | | | | |
|---|---|---|---|---|---|---|---|---|
| | | | Iso- | Addi- | Storage period | | | |
| | | | electric | tion of | Begin- | 1st | 2nd | 4^{th} |
| | Grade | pH | point | sorbitol | ning | week | week | week |
| Sample | SF | 5.7 | 3.1 | 20 g | − | − | − | − |
| B8 | | | | | | | | |

| | | | | | | |
|---|---|---|---|---|---|---|
| Sample | No addition | 40 g | − | − | + | + |
| B15 | | | | | | |

−: Clear liquid, or negligible precipitation or opacity occurs
+: Precipitation or opacity occurs
++: Conspicuous precipitation or opacity occurs

What is claimed is:

1. A liquid preparation containing a herb-origin extract having pH of 3.0 to 5.5 and containing the herb-origin extract at a concentration of 5 to 50 w/v % (dry weight) to the total amount of the liquid preparation, comprising a caramel at a concentration of 0.5 to 20 w/v % to the total amount of the liquid preparation, wherein the caramel has an isoelectric point of 2.5 to 4.0 and a 1.0 w/v % aqueous solution of the caramel has a pH of 4.5 to 6.0.

2. The liquid preparation as set forth claim 1, wherein the herb-origin extract comprises one or more extracts selected from those extracted from an Epimedium grandiflorum, an Acanthopanax senticosus, a Poria Sclerotium, a Rehmannia Root, a Polygonum Root, a Cuscuta Seed, a Garlic, a Ginseng and a Ginkgo.

3. The liquid preparation as set forth claim 1 or 2, comprising an animal-origin extract at percentage between 5 to 60 w/v % to the herb-origin extract.

4. The liquid preparation as set forth any one of claims 1 to 3, wherein the herb-origin extract comprises at least the Garlic extract.

5. The liquid preparation as set forth any one of claims 1 to 4, further comprising a monosaccharide and/or sugar alcohol at concentration between 1.0 and 40 w/v % to the total amount of the liquid preparation.

6. A method of stabilizing a liquid preparation containing a herb-origin extract having a pH of 3.0 to 5.5 and containing the herb-origin extract at a concentration of 5 to 50 w/v % (dry weight) to the total amount of the liquid preparation; comprising a caramel at a concentration of 0.5 to 20 w/v % to the total amount of the liquid preparation, wherein the caramel has an isoelectric point of 2.5 to 4.0 and a 1.0 w/v % aqueous solution of the caramel has pH of 4.5 to 6.0.

7. A liquid preparation containing an animal-origin extract having a pH of 3.0 to 5.5 and containing the animal-origin extract at a concentration of 5 to 50 w/v % (dry weight) to the total amount of the liquid preparation; comprising a caramel at a concentration of 0.5 to 20 w/v % to the total amount of the liquid preparation, wherein the caramel has an isoelectric point of 0.5 to 4.0 and a 1.0 w/v % aqueous solution of the caramel has a pH of 4.5 to 6.1.

8. The liquid preparation as set forth claim 7, wherein the animal-origin extract comprises at least one or more extracts selected from those extracted from an Oriental Bezoar, a Civet, a Royal Jelly, a Deer Velvet Antler and a liver hydrolysate.

9. The liquid preparation as set forth claim 7 or 8, further comprising a monosaccharide and/or sugar alcohol at a concentration between 1.0 and 40 w/v % to the total amount of the liquid preparation.

10. A method of stabilizing a liquid preparation containing an animal-origin extract having a pH of 3.0 to 5.5 and containing the animal-origin extract at a concentration of 5 to 50 w/v % (dry weight) to the total amount of the liquid preparation; comprising a caramel at a concentration of 0.5 to 20 w/v % to the total amount of the liquid preparation, wherein the caramel has an isoelectric point of 0.5 to 4.0 and a 1.0% aqueous solution of the caramel has a pH of 4.5 to 6.1.
